(19) United States
(12) Patent Application Publication
(10) Pub. No.: US 2022/0370238 A1
(43) Pub. Date: Nov. 24, 2022

(54) MENSTRUAL CUP

(71) Applicant: Ki Joon PARK, Seoul (KR)

(72) Inventor: Ki Joon PARK, Seoul (KR)

(73) Assignee: STANDARDPARK CO., LTD., Seoul, 05719 (KR)

(21) Appl. No.: 17/296,246

(22) PCT Filed: Nov. 21, 2019
(86) PCT No.: PCT/KR2019/016052

§ 371 Date: Oct. 12, 2021

(30) Foreign Application Priority Data
Feb. 14, 2019 (KR) 10-2019-0017067

(51) Int. Cl. A61F 5/455; A61F 5/44; A61F 5/441

(52) U.S. Cl. CPC A61F 5/4553; A61F 5/4401; A61F 5/441; A61F 5/4408

(57) ABSTRACT

A menstrual cup is capable of having various sizes customizedly selected according to the characteristics of women's vaginas having different sizes by providing an upper restoring ring having a diameter larger than the diameter of a restoring ring provided on a flexible cup, so that if the menstrual cup is inserted into a wearer's vagina to cause her to feel painful because it is bigger than her vagina, the upper restoring ring is removed, and next, the menstrual cup with the restoring ring smaller than the upper restoring ring is inserted into the vagina, thereby achieving pain removal and safe and convenient collection of menstrual blood and perfectly preventing the initial reluctance of women in the use of the menstrual cup in spite of many advantages the menstrual cup has had.

### BACKGROUND AND SUMMARY

The present invention relates to a menstrual cup, and more particularly, to a menstrual cup that is capable of having various sizes customizedly selected, according to the characteristics of women's vaginas having different sizes, by providing an upper restoring ring having a diameter larger than the diameter of a restoring ring, so that if the menstrual cup is inserted into a wearer's vagina to cause her to feel painful because it is bigger than her vagina, the upper restoring ring is removed, and next, the menstrual cup with the restoring ring smaller than the upper restoring ring is inserted into the vagina, thereby achieving pain removal and safe and convenient collection of menstrual blood.

Generally, menstrual products for women include sanitary pads attached to the inside of the underwear (panties) and insertion type tampons inserted into the vagina.

The sanitary pad advantageously has an excellent absorption force and a low thickness, but while a woman who wears the sanitary pad on her panties is moving her body, the sanitary pad may move easily, thereby having limitations in her motions or exercise. Even during sleep, further, she has to be careful of her motion. Above all, the surface of the sanitary pad, which comes into contact with the skin during the wearing, is made of a chemical material, thereby causing skin diseases or itching.

The insertion type tampon prevents menstrual blood from leaking therefrom in a more effective manner than the sanitary pad, thereby ensuring the wearer's free activity during menstruation, but as the insertion type tampon is made of chemical components, unfortunately, the harmful materials generated therefrom may cause various diseases.

In specific, the sanitary pad and the insertion type tampon are disposable products that are hard to be reusable, thereby causing environmental pollution and economical load due to high treatment cost for large amounts of waste discharged.

So as to solve such problems the sanitary pad and the insertion type tampon have had, recently, an insertion type menstrual cup, which is reusable because it is made of a silicone material and is designed to collect menstrual blood by the insertion into a woman's vagina, has been proposed to ensure her free activity.

FIG. 1a is a perspective view showing an insertion type menstrual cup in the conventional practice (as disclosed in Korean Patent No. 1915316), FIG. 1b is a sectional view taken along the line A-A of FIG. 1a, and FIG. 1c is a perspective view showing a process where the conventional insertion type menstrual cup is used.

As shown in FIGS. 1a to 1c, the conventional insertion type menstrual cup includes a body 1 adapted to be inserted into a woman's vagina to collect menstrual blood therein, the body 1 having a shape of a cup open on top thereof in such a manner as to become reduced in inner diameter from top toward bottom, and a grip 2 extended downward from the underside of the body 1 to easily draw the body 1 inserted into the vagina to the outside. In this case, the body 1 is made of silicone containing phytoncide and has an absorbing material 10 built in the bottom surface thereof to absorb the menstrual blood, thereby preventing the menstrual blood stored in the body 1 from flowing back into the vagina. Further, the body 1 has a restoring ring 30 built in the top periphery thereof to allow the top deformed to be easily restored to its original shape and a plurality of deformation inducing sections 31 spaced apart from each other at equal angles to each other and having a low thickness.

According to the conventional insertion type menstrual cup, the body 1, which is inserted into the vagina, is made of the sanitary silicone containing the phytoncide having antibacterial effectiveness, thereby minimizing the infection of the vagina resulting from the invasion of harmful bacteria to allow the insertion type menstrual cup to be reliably used. Further, the absorbing material disposed in the body 1 prevents the menstrual blood stored in the body 1 from flowing back into the vagina, thereby suppressing abdominal pain and various diseases from occurring due to the backflow of the menstrual blood.

However, women's vaginas have different sizes according to their age ranged from the elementary school girls with menarche to the women going through menopause who in their mid and late 40s, especially according to their delivery experience and body shape, and if the menstrual cup which does not correspond to the wearer's vaginal size is inserted into the vagina, in this case, pain may occur or menstrual blood may leak frequently.

In spite of the menstrual cup having many advantages, accordingly, women are reluctant to use the menstrual cup, and otherwise, they inconveniently have to find the menstrual cup corresponding to their vaginal size by trying a number of menstrual cups.

Accordingly, the present invention has been made in view of the above-mentioned problems occurring in the related art, and it desirable to provide a menstrual cup that is capable of having various sizes customizedly selected, according to the characteristics of women's vaginas having different sizes, by providing an upper restoring ring having a diameter larger than the diameter of a restoring ring, so that if the menstrual cup is inserted into a wearer's vagina to cause her to feel painful because it is bigger than her vagina, the upper restoring ring is removed, and next, the menstrual cup with the restoring ring smaller than the upper restoring ring is inserted into the vagina, thereby achieving pain removal and safe and convenient collection of menstrual blood and perfectly preventing the initial reluctance of women in the use of the menstrual cup in spite of many advantages the menstrual cup has had.

It is also desirable to provide a menstrual cup that is capable of being variable in size by providing a restoring ring, an upper restoring ring, and an uppermost restoring ring, so that in consideration of pain occurrence and leakage of menstrual blood after the insertion into the vagina, accordingly, if the vaginal size is relatively large, the menstrual cup with the uppermost restoring ring is used, if pain or unpleasant feeling occurs during the use of the uppermost restoring ring, the menstrual cup with the upper restoring ring after the uppermost restoring ring has been removed is used, and if pain still occurs even during the use of the upper restoring ring, the menstrual cup just with the restoring ring is used, thereby allowing the menstrual cup conveniently fitted to a wearer's vagina to be customizedly used.

It is also desirable to provide a menstrual cup that is capable of being provided with a single restoring ring after the size thereof is customizedly selected by a wearer, so that the wearer just purchases the menstrual cup with the single restoring ring, which corresponds to her vaginal size, thereby saving the purchase cost and in advance preventing environmental pollution from occurring.

It is also desirable to provide a menstrual cup that is capable of allowing, if it is desired to remove an upper restoring ring or uppermost restoring ring, the upper restoring ring or the uppermost restoring ring to be easily and cleanly separated along an upper cut-off groove or uppermost cut-off groove.

It is also desirable to provide a menstrual cup that is capable of allowing, if it is desired to remove an uppermost restoring ring, the uppermost restoring ring to be easily and cleanly separated along an uppermost cut-off groove concaved inward from the boundary line between the end line of the horizontal top periphery of an upper restoring ring and the start line of the rising bottom periphery of the uppermost restoring ring, and allowing, if it is desired to remove the upper restoring ring, the upper restoring ring to be easily and cleanly separated along an upper cut-off groove concaved inward from the boundary line between the end line of the horizontal top periphery of a restoring ring and the start line of the rising bottom periphery of the upper restoring ring, so that after the removal of the uppermost restoring ring or the upper restoring ring, accordingly, the menstrual cup can be maintained with a smoothly curved surface, thereby making a wearer feel no displeasure when the insertion of the menstrual cup into her vagina.

It is also desirable to provide a menstrual cup that is capable of allowing an upper cut-off groove and an uppermost cut-off groove to be open to the outside to the maximum extent, and simultaneously, allowing the restoring forces of an upper restoring ring and an uppermost restoring ring to be ensured by means of an upper extension volume portion and an uppermost extension volume portion, thereby being usable more cleanly and conveniently.

According to an aspect of the present invention, there is provided a menstrual cup having a flexible cup adapted to be inserted into a woman's vagina to collect menstrual blood therein, a restoring ring provided on top periphery of the flexible cup, and a grip provided on underside of the flexible cup, the menstrual cup comprising an upper restoring ring protruding upward and outward from an upper extension line extended upward from the restoring ring in such a manner as to have a diameter larger than the diameter of the restoring ring.

According to an aspect of the present invention, the menstrual cup can have various sizes customizedly selected according to the characteristics of women's vaginas having different sizes, and to do this, the upper restoring ring is provided to have a diameter larger than the diameter of the restoring ring, so that if the menstrual cup is inserted into a wearer's vagina to cause the wearer to feel painful because it is bigger than her vagina, the upper restoring ring is removed, and next, the menstrual cup with the restoring ring smaller than the upper restoring ring is inserted into the vagina, thereby achieving pain removal and safe and convenient collection of menstrual blood and perfectly preventing the initial reluctance of women in the use of the menstrual cup in spite of many advantages the menstrual cup has had.

According to an aspect of the present invention, further, the menstrual cup is variable in size by providing the restoring ring, the upper restoring ring, and the uppermost restoring ring, and in consideration of pain occurrence and leakage of menstrual blood after the insertion of the menstrual cup into the vagina, accordingly, if the vaginal size is relatively large, the menstrual cup with the uppermost restoring ring is used, if pain or unpleasant feeling occurs during the use of the uppermost restoring ring, the menstrual cup with the upper restoring ring after the uppermost restoring ring has been removed is used, and if pain still occurs even during the use of the upper restoring ring, the menstrual cup just with the restoring ring is used, thereby allowing the menstrual cup conveniently fitted to a wearer's vagina to be customizedly used.

In addition, if the menstrual cup corresponding to the wearer's vaginal size is found, the menstrual cup with the single restoring ring, which corresponds to her vaginal size, is purchased, thereby saving the purchase cost and in advance preventing environmental pollution from occurring.

If it is desired to remove the upper restoring ring or the uppermost restoring ring, also, the upper restoring ring or the uppermost restoring ring can be easily and cleanly separated along the upper cut-off groove or the uppermost cut-off groove.

If it is desired to remove the uppermost restoring ring, further, the uppermost restoring ring can be easily and cleanly separated along the uppermost cut-off groove concaved inward from the boundary line between the end line of the horizontal top periphery of the upper restoring ring and the start line of the rising bottom periphery of the uppermost restoring ring, and if it is desired to remove the upper restoring ring, the upper restoring ring can be easily and cleanly separated along the upper cut-off groove concaved inward from the boundary line between the end line of the horizontal top periphery of the restoring ring and the start line of the rising bottom periphery of the upper restoring ring, so that after the removal of the uppermost restoring ring or the upper restoring ring, the menstrual cup can be maintained with a smoothly curved surface, thereby making a wearer feel no displeasure when the insertion of the menstrual cup into her vagina.

Moreover, the upper cut-off groove and the uppermost cut-off groove are open to the outside to the maximum extent, and simultaneously, the restoring forces of the upper restoring ring and the uppermost restoring ring can be ensured by means of the upper extension volume portion and the uppermost extension volume portion, thereby allowing the menstrual cup to be usable more cleanly and conveniently.

Also, if it is desired to separate the menstrual cup in which the menstrual blood is collected from the vagina, air is introduced or exhausted through the air holes to allow the menstrual cup to be released from the vacuum state, thereby performing the separation of the menstrual cup in easier and simpler manners.

### BRIEF DESCRIPTION OF DRAWINGS

FIG. 1a is a perspective view showing a conventional insertion type menstrual cup.

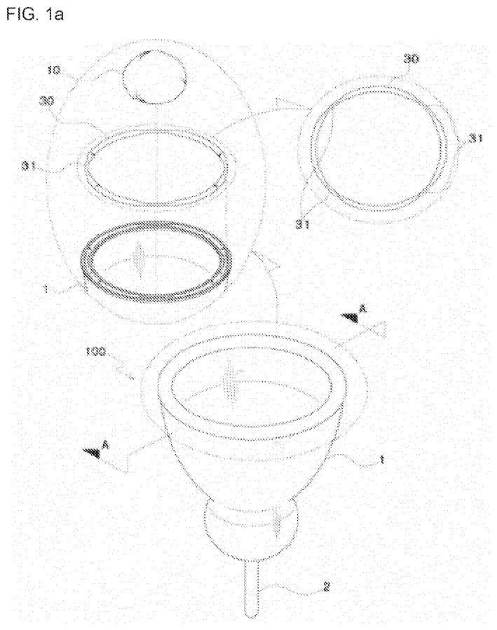

FIG. 1b is a sectional view taken along the line A-A of FIG. 1a.

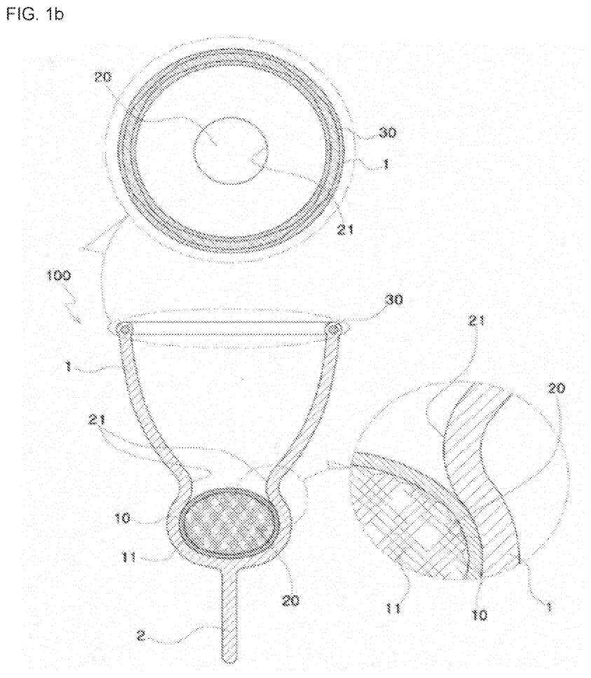

FIG. 1c is a perspective view showing a process where the conventional insertion type menstrual cup is used.

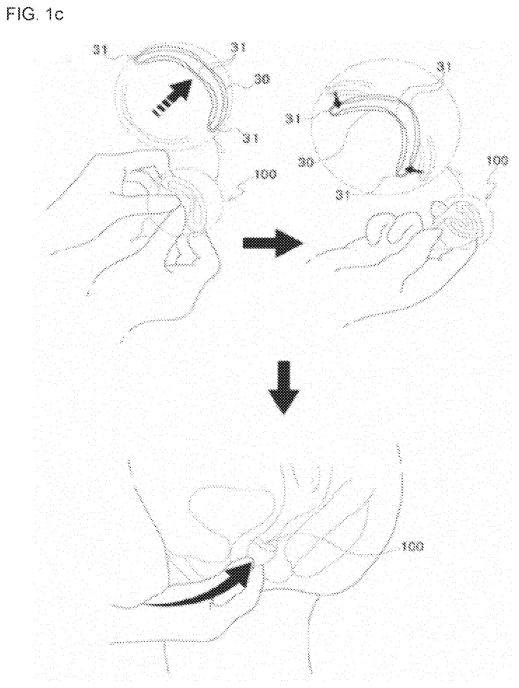

FIG. 2 is a perspective view showing a menstrual cup according to the present invention.

FIG. 3 is a sectional view showing the menstrual cup according to the present invention.

FIG. 4 is a perspective view showing a process where the menstrual cup according to the present invention is used.

### DETAILED DESCRIPTION

According to the best mode for the present invention, there is provided a menstrual cup having a flexible cup 50 adapted to be inserted into a woman's vagina to collect menstrual blood therein, a restoring ring 60 provided on top periphery of the flexible cup 50, and a grip 90 provided on underside of the flexible cup 50, the menstrual cup including an upper restoring ring 70 protruding upward and outward from an upper extension line 71 extended upward from the restoring ring 60 in such a manner as to have a diameter larger than the diameter of the restoring ring 60.

Hereinafter, an explanation of the present invention will be in detail given with reference to the attached drawings. Objects, characteristics and advantages of the present invention will be more clearly understood from the detailed description as will be described below and the attached drawings.

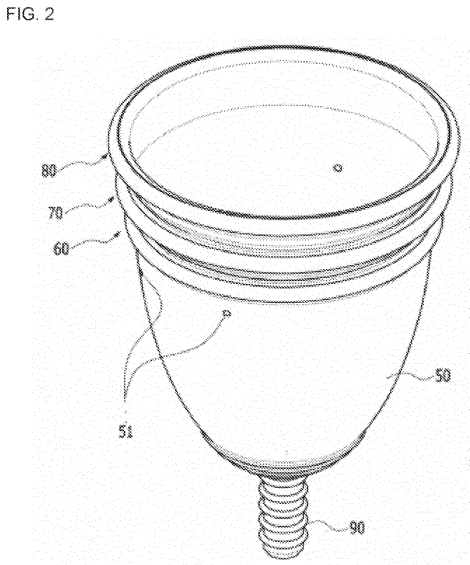

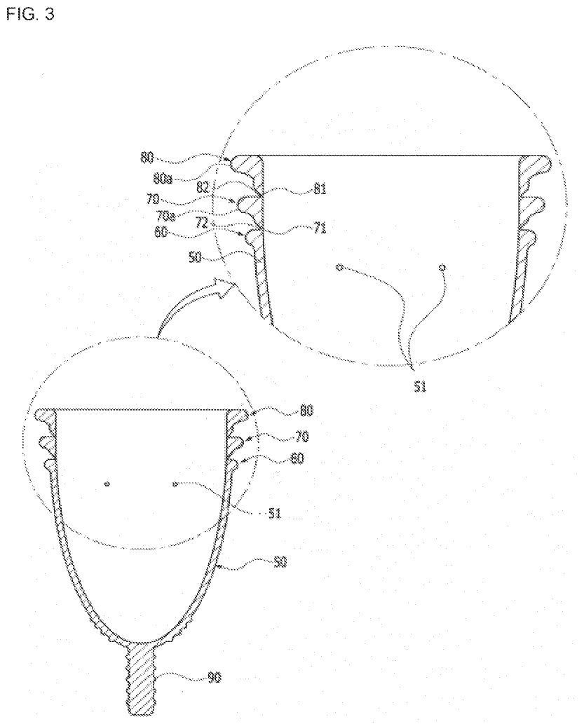

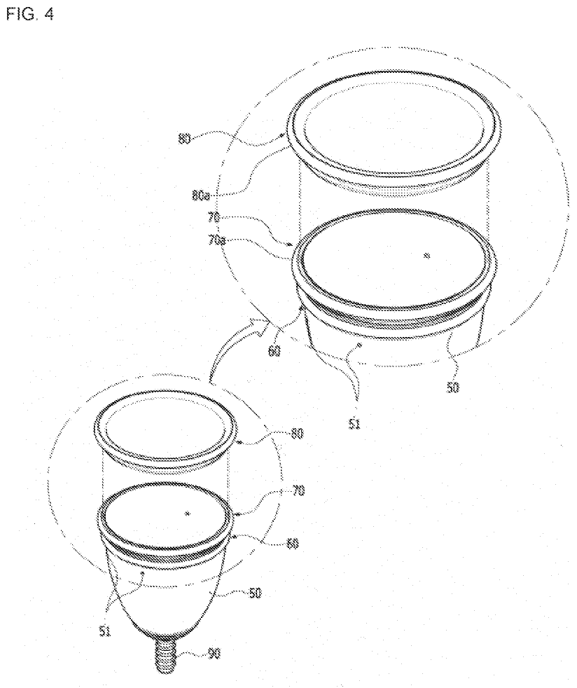

FIG. 2 is a perspective view showing a menstrual cup according to the present invention, FIG. 3 is a sectional view showing the menstrual cup according to the present invention, and FIG. 4 is a perspective view showing a process where the menstrual cup according to the present invention is used.

As shown in FIGS. 2 to 4, the menstrual cup according to the present invention includes a flexible cup 50 adapted to be inserted into a woman's vagina to collect menstrual blood therein, a restoring ring 60 provided on top periphery of the flexible cup 50, and a grip 90 provided on underside of the flexible cup 50.

The menstrual cup, which is inserted into the woman's vagina, is made of a silicone material that is not harmful to the human body, is easy to be bent, and is excellent in restoration, and if the menstrual cup is inserted into the vagina in a state of being folded, it is unfolded by means of the restoring ring 60 to allow the menstrual blood discharged from the well to be safely collected in the flexible cup 50.

If it is desired to separate the menstrual cup in which the menstrual blood is filled from the vagina, the grip 90 is held to draw the menstrual cup from the vagina, and after the menstrual blood is removed, next, the menstrual cup is washed and inserted into the vagina again. During the menstrual period of the woman, accordingly, the menstrual cup can be repeatedly used in safe and convenient manners, without having any problems like environmental pollution caused by general sanitary pads and insertion type tampons.

According to the present invention, the menstrual cup includes an upper restoring ring 70 protruding upward and outward from an upper extension line 71 extended upward from the restoring ring 70 in such a manner as to have a diameter larger than the diameter of the restoring ring 60.

Women's vaginas have different sizes according to their age, delivery experience, and body shape, and if the menstrual cup corresponding to the vaginal size is not used, accordingly, problems like pain occurrence or menstrual blood leakage may occur. According to the present invention, the menstrual cup can have various sizes customizedly selected according to the characteristics of the women's vaginas having different sizes, and to do this, the upper restoring ring 70 is provided to have a diameter larger than the diameter of the restoring ring 60. If the menstrual cup is inserted into the vagina to cause a wearer to feel painful because it is bigger than her vagina, the upper restoring ring 70 is removed, and next, the menstrual cup with the restoring ring 60 smaller than the upper restoring ring 70 is inserted into the vagina, thereby achieving pain removal and safe and convenient collection of menstrual blood and perfectly preventing the initial reluctance of the women in the use of the menstrual cup in spite of many advantages the menstrual cup has had.

According to the present invention, desirably, the menstrual cup includes an uppermost restoring ring 80 protruding upward and outward from an uppermost extension line 81 extended upward from the upper restoring ring 70 in such a manner as to have a diameter larger than the diameter of the upper restoring ring 70.

It is hard for a woman who first uses the menstrual cup to find the menstrual cup with a size corresponding to her vaginal size, and according to the present invention, the menstrual cup is variable in size by providing the restoring ring 60, the upper restoring ring 70, and the uppermost restoring ring 80, so that in consideration of pain occurrence, unpleasant feeling, and leakage of menstrual blood after the insertion of the menstrual cup into the vagina, if the vaginal size is relatively large, the menstrual cup with the uppermost restoring ring 80 is used, if pain or unpleasant feeling occurs during the use of the uppermost restoring ring 80, the menstrual cup with the upper restoring ring 70 after the uppermost restoring ring 80 has been removed is used, and if pain still occurs even during the use of the upper restoring ring 70, the menstrual cup just with the restoring ring 80 is used, thereby allowing the menstrual cup conveniently fitted to a wearer to be used customizedly.

According to the present invention, in this case, the menstrual cup includes the restoring ring 60, the upper restoring ring 70, and the uppermost restoring ring 80, but of course, the number of restoring rings may be added. Further, if the menstrual cup corresponding to the wearer's vaginal size is found, the menstrual cup with the single restoring ring 60, which corresponds to her vaginal size, is purchased, thereby saving the purchase cost and in advance preventing environmental pollution from occurring.

Further, the menstrual cup according to the present invention has an upper cut-off groove 72 formed inward from the boundary line between the restoring ring 60 and the upper restoring ring 70 and an uppermost cut-off groove 82 formed inward along the boundary line between the upper restoring ring 70 and the uppermost restoring ring 80, so that if it is desired to remove the upper restoring ring 70 or the uppermost restoring ring 80, the upper restoring ring 70 or the uppermost restoring ring 80 can be easily and cleanly taken off along the upper cut-off groove 72 or the uppermost cut-off groove 82 from the restoring ring 60 or the upper restoring ring 70.

Moreover, the upper cut-off groove 72 is concaved inward from the boundary between the end line of the horizontal top periphery of the restoring ring 60 and the start line of the rising bottom periphery of the upper restoring ring 70, and the uppermost cut-off groove 82 is concaved inward from the boundary between the end line of the horizontal top periphery of the upper restoring ring 70 and the start line of the rising bottom periphery of the uppermost restoring ring 80.

If it is desired to remove the uppermost restoring ring 80, the uppermost restoring ring 80 can be easily and cleanly separated from the upper restoring ring 70 along the uppermost cut-off groove 82 concaved inward from the boundary between the end line of the horizontal top periphery of the upper restoring ring 70 and the start line of the rising bottom periphery of the uppermost restoring ring 80, and if it is desired to remove the upper restoring ring 70, the upper restoring ring 70 can be easily and cleanly separated from the restoring ring 60 along the upper cut-off groove 72 concaved inward from the boundary between the end line of the horizontal top periphery of the restoring ring 60 and the start line of the rising bottom periphery of the upper restoring ring 70. After the removal of the uppermost restoring ring 80 or the upper restoring ring 70, accordingly, the menstrual cup can be maintained with a smoothly curved surface, thereby making a wearer feel no displeasure when the insertion of the menstrual cup into her vagina.

On the other hand, the upper restoring ring 70 has an upper extension volume portion 70a gently raised from the upper cut-off groove 72 and then protruding outward therefrom with a given volume, and the uppermost restoring ring 80 has an uppermost extension volume portion 80a gently raised from the uppermost cut-off groove 72 and then protruding outward therefrom with a given volume, so that the upper cut-off groove 72 and the uppermost cut-off groove 82 are open to the outside to the maximum extent, and simultaneously, the restoring forces of the upper restoring ring 70 and the uppermost restoring ring 80 can be ensured by means of the upper extension volume portion 70a and the uppermost extension volume portion 80a, thereby allowing the menstrual cup to be usable more cleanly and conveniently.

On the other hand, the menstrual cup according to the present invention further includes a plurality of air holes 51 formed on the upper periphery of the flexible cup 50, so that if it is desired to separate the menstrual cup in which the menstrual blood is collected from the vagina, air is introduced or exhausted through the air holes 51 to allow the flexible cup 50 to be released from the vacuum state, thereby performing the separation of the menstrual cup in easier and simpler manners.

The present invention can be applied to the industrial field in which woman's menstrual blood is easily and conveniently treated, while no environmental pollution is occurring.

1. A menstrual cup having a flexible cup adapted to be inserted into a woman's vagina to collect menstrual blood therein, a restoring ring provided on top periphery of the flexible cup, and a grip provided on underside of the flexible cup, the menstrual cup comprising an upper restoring ring protruding upward and outward from an upper extension line extended upward from the restoring ring in such a manner as to have a diameter larger than the diameter of the restoring ring, the upper restoring ring comprises an upper cut-off groove formed inward from the boundary with the restoring ring.

2. The menstrual cup according to claim 1, further comprising an uppermost restoring ring protruding upward and outward from an uppermost extension line extended upward from the upper restoring ring in such a manner as to have a diameter larger than the diameter of the upper restoring ring, the uppermost restoring ring comprising an uppermost cut-off groove formed inward along the boundary with the upper restoring ring.

3. (canceled)

4. The menstrual cup according to claim 3, wherein the upper cut-off groove is concaved inward from the boundary between the end line of the horizontal top periphery of the restoring ring and the start line of the rising bottom periphery of the upper restoring ring, and the uppermost cut-off groove is concaved inward from the boundary between the end line of the horizontal top periphery of the upper restoring ring and the start line of the rising bottom periphery of the uppermost restoring ring.

5. The menstrual cup according to claim 4, wherein the upper restoring ring has an upper extension volume portion gently raised from the upper cut-off groove and then protruding outward therefrom with a given volume, and the uppermost restoring ring has an uppermost extension volume portion gently raised from the uppermost cut-off groove and then protruding outward therefrom with a given volume.

6. The menstrual cup according to claim 1, further comprising a plurality of air holes formed on the upper periphery of the flexible cup.
